(19) United States
(12) Patent Application Publication
(10) Pub. No.: US 2020/0392439 A1
(43) Pub. Date: Dec. 17, 2020

(54) ARRAY AND USE THEREOF

(71) Applicant: The University of Tokyo, Tokyo (JP)

(72) Inventors: Shoji Takeuchi, Tokyo (JP); Haruka Yoshie, Tokyo (JP); Ai Shima, Tokyo (JP); Shogo Nagata, Tokyo (JP)

(73) Assignee: The University of Tokyo, Tokyo (JP)

(21) Appl. No.: 16/957,037

(22) PCT Filed: Nov. 9, 2018
(86) PCT No.: PCT/JP2018/041720

§ 371 Date: Aug. 18, 2020

Related U.S. Application Data
(60) Provisional application No. 62/610,160, filed on Dec. 23, 2017.

(51) Int. Cl. C12M 1/12; C12N 11/04; C12M 1/00; C12M 3/00

(52) U.S. Cl. CPC C12M 23/06; C12N 11/04; C12M 25/10; C12M 23/20; C12M 23/42; C12M 23/52

(57) ABSTRACT

An array includes a plurality of tubes disposed in contact with each other such that axial directions are parallel to each other; and a target substance disposed inside at least one of the plurality of tubes, in which the plurality of tubes are made of anionic hydrogel, and surfaces on which the plurality of tubes are in contact with each other are bonded with an adhesive including nanoparticles having surfaces coated with a cationic water-soluble polymer.

### TECHNICAL FIELD

The present invention relates to an array and a use thereof. More particularly, the present invention relates to an array, a cell culture method, a method for transporting non-frozen cells alive, and a method for manufacturing an array. Priority is claimed on U.S. Pat. No. 62/610,160 provisionally filed in United States, filed Dec. 23, 2017, the content of which is incorporated herein by reference.

### BACKGROUND ART

An array has target substances disposed on a substrate. For example, a cell array, a DNA array, a protein array and the like are known. For example, Patent Literature 1 discloses the use of cell arrays for screening of test compounds, a toxicological assay, a single cell differentiation study, a cell function study, and the like. Patent Literature 1 also discloses a cell array in which a plurality of independent spots are spotted on an upper surface of a chemically modified glass slide, each spot includes a matrix bottom layer and a matrix surface layer, and the matrix surface layer includes cells.

### CITATION LIST

### Patent Literature

### [Patent Literature 1]

Japanese Translation of PCT International Application Publication No. JP-T-2009-513160

### SUMMARY OF INVENTION

### Technical Problem

However, the cell array disclosed in Patent Literature 1 needs to spot cells in a manufacturing process. Therefore, for example, when producing a cell array made up of a plurality of types of cells, there are cases in which one spot is mixed with heterogeneous cells. In addition, when producing an array of cell aggregates such as spheroids, it takes a long time for spheroids to form. An object of the present invention is to provide a new array.

### Solution to Problem

The present invention includes the following aspects.

[1] An array including: a plurality of tubes disposed in contact with each other such that axial directions thereof are parallel to each other; and a target substance disposed inside at least one of the plurality of tubes, in which the plurality of tubes are made of anionic hydrogel, and surfaces on which the plurality of tubes are in contact with each other are bonded with an adhesive including nanoparticles having surfaces coated with a cationic water-soluble polymer.

[2] The array described in [1], in which some of the plurality of tubes may include the target substance inside, and the remaining tubes include no target substance.

[3] The array described in [1] or [2], in which the target substance may be a cell.

[4] The array described in [3], in which the cells may include a cell which respond to a chemical substance.

[5] The array described in [3] or [4], in which cells may be disposed inside two adjacent tubes of the plurality of tubes, and tubes around the two tubes include no cells.

[6] A cell culture method including: incubating the array described in [5] in a culture medium, wherein, as a result, the cells located inside two adjacent tubes each grow and come into contact with each other to form a contact surface.

[7] A method for transporting non-frozen cells alive, the method including: transporting a container which accommodates the array described in any one of [3] to [5] and the culture medium.

[8] A method for manufacturing an array, the method including: disposing a plurality of fibers including a target substance inside a tube made of anionic hydrogel to be in contact with each other so that axial directions are parallel to each other, and bonding the fibers with an adhesive including nanoparticles having a surface coated with cationic water-soluble polymer to obtain a bundle of the fibers; embedding the bundle in a support material; and cutting the bundle together with the support material to obtain a section, in which the section is an array.

[9] The method for manufacturing an array described in [8], in which a quotient (a storage elastic modulus/a loss elastic modulus) obtained by dividing a storage elastic modulus of the support material by a loss elastic modulus is larger than 10, and the storage elastic modulus is 100 kPa or less.

[10] The method for manufacturing an array described in [8] or [9], in which the fiber is manufactured by a process of introducing the target substance through a funnel-type device into a tube made of anionic hydrogel.

[11] The method for manufacturing an array described in any of [8] to [10], in which the target substance is a cell.

### Advantageous Effects of Invention

According to the present invention, a new array can be provided.

### BRIEF DESCRIPTION OF DRAWINGS

FIG. 1 is a schematic view showing an example of a structure of an array.

FIG. 2 is a schematic view showing an example of a method for manufacturing a fiber.

FIG. 3 is a schematic view showing an example of a method for manufacturing an array.

FIG. 4 is a graph showing results of Experimental Example 1.

FIG. 5 is a photograph of a fluorescent bead array observed by a fluorescent microscope in experimental example 2.

FIG. 6 is a photograph of the fluorescent bead array observed by the fluorescent microscope in experimental example 2.

FIG. 7(a) is a photograph showing a result of detecting live cells in a cell array in experimental example 2. FIG. 7(b) is a photograph showing a result of detecting dead cells in the cell array in experimental example 2. FIG. 7(c) is a photograph showing a result of detecting nuclei in the cell array in experimental example 2. FIG. 7(d) is a photograph obtained by merging (a) to (c).

FIG. 8(a) is a fluorescence microphotograph obtained by photographing fluorescence of a calcium probe in experimental example 3. FIG. 8(b) is a graph showing a result of time-dependent measurement of a fluorescence intensity of the calcium probe after addition of muscarine in experimental example 3.

FIG. 9(a) is a microphotograph of the cell array before transportation observed in experimental example 4. FIG. 9(b) is a microphotograph of the cell array after transportation observed in experimental example 4. FIG. 9(c) is a photograph showing a result of detecting live cells after transportation in experimental example 4. FIG. 9(d) is a photograph showing a result of detecting dead cells after transportation in experimental example 4.

### DESCRIPTION OF EMBODIMENTS

Hereinafter, embodiments of the present invention will be described in detail while referring to the drawings in some cases. In the drawings, the same or corresponding parts are denoted by the same or corresponding reference numerals, and repeated description will be omitted. The dimensional ratios in each drawings are exaggerated for the sake of explanation, and do not necessarily match the actual dimensional ratios.

### [Array]

In an embodiment, the present invention provides an array which includes a plurality of tubes disposed in contact with each other such that axial directions are parallel to each other, and a target substance disposed inside at least one of the plurality of tubes, in which the plurality of tubes are made of anionic hydrogel, and surfaces on which the plurality of tubes are in contact with each other are bonded with an adhesive including nanoparticles having surfaces coated with a cationic water-soluble polymer.

The target substance is not particularly limited, and examples thereof include animal cells, plant cells, cells such as microorganisms, biomolecules such as a DNA, RNA and proteins, tissue fragments derived from a living body, a compound such as a low molecular weight compound, and the like.

The compound includes, for example, drugs. The compound may be a library. Examples thereof include natural compound libraries, synthetic compound libraries, existing drug libraries, metabolite libraries, and the like.

For example, when the target substance is DNA, the array of this embodiment can be used as a DNA array. Similarly, when the target substance is a protein, the array of this embodiment can be used as a protein array. Hereinafter, an array in which the target substance is a cell may be particularly referred to as a “cell array”.

For example, cell aggregates such as spheroids, which have a three-dimensional cell structure, are closer to living organisms than two-dimensional cultured cells, and are therefore expected to be increasingly used in drug screening or toxicity tests. The array of this embodiment can be used as a cell aggregate array and can be said to be a highly biomimetic three-dimensional tissue array. As will be described later, the array of this embodiment can be used as a chemical substance sensor, a transporting means of cells, an efficient drug screening system, etc., and can also be used for a cell test in a place where cell culturing is difficult, such as in the field.

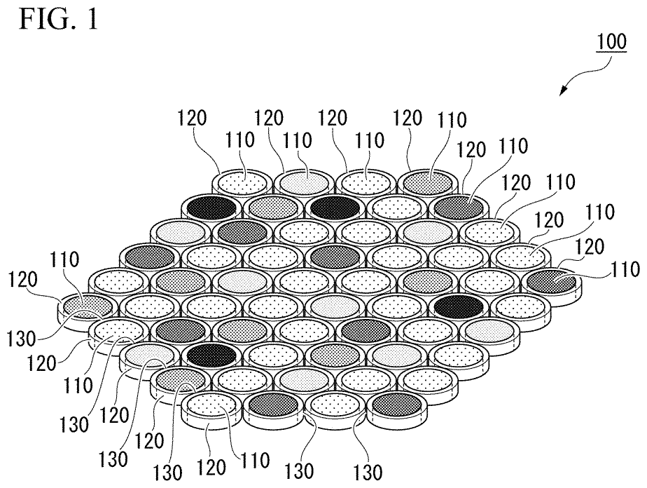

FIG. 1 is a schematic view showing a structure of the array of this embodiment. As shown in FIG. 1, an array 100 includes a plurality of tubes 120 disposed in contact with each other such that axial directions are parallel to each other, and a target substance 110 disposed in at least one of the plurality of tubes 120. The plurality of tubes 120 are made of anionic hydrogel, and a surface 130 on which the plurality of tubes 120 are in contact with each other is bonded with an adhesive including nanoparticles having surfaces coated with a cationic water-soluble polymer. In the present specification, the axial direction of the tube means a direction along a central axis of the tube.

When the target substance is a cell, a thickness of the array is preferably 800 or less, more preferably about 200 μm, from the viewpoint of supplying oxygen or nutrients to the cell.

A hydrogel is a three-dimensional network structure including a large amount of water. In the array of this embodiment, an alginic acid hydrogel can be suitably used as the anionic hydrogel. An alginic acid hydrogel means a hydrogel obtained by forming a salt of alginic acid and a divalent metal ion (calcium ion, barium ion, etc.).

Since anionic hydrogels hydrate in an aqueous solvent such as water, culture medium, buffer solution, etc., it is difficult to keep an adhesion state of anionic hydrogel in the aqueous solvent. In contrast, the inventors found that anionic hydrogel can be bonded together with an adhesive including nanoparticles in which a surface is coated with a cationic water-soluble polymer (hereinafter sometimes referred to as “CNP”). Anionic hydrogel bonded by CNP can maintain a stable adhesion state even in an aqueous solvent. Further, CNP has almost no toxicity to cells. The adhesive including CNP will be described below.

The type of target substance contained in the array of this embodiment is arbitrary and may be of one or more types. Further, the array of this embodiment may include the target substance in all of the plurality of tubes. Alternatively, some of the plurality of tubes may contain the target substance inside, and the remaining tubes may not contain the target substance. An arrangement of the target substance can be arbitrarily controlled, by controlling the arrangement of the tubes including the target substance and the tubes not including the target substance. For example, in the example of the array of FIG. 1, although the target substance is not disposed in a grid shape, the target substance can be disposed in a grid shape, by controlling the arrangement of the tubes including the target substance and the tubes not including the target substance.

In the array of this embodiment, the target substance may be cells. Further, at least some of the cells may be cells that respond to a chemical substance. A chemical substance sensor using cells is useful because of its high sensitivity and high selectivity. In recent years, with the development of gene transfer technology, it has become possible to express a receptor for an arbitrary chemical substance on the cell membrane surface, and attention to chemical substances sensor using cells has increased further.

As described below in the examples, for example, by reacting muscarine with a cell array in which cells expressing muscarinic acetylcholine receptors are placed, inflow of calcium into cells can be detected.

The array of this embodiment may be one in which cells are disposed inside two adjacent tubes of a plurality of tubes, with tubes around the two tubes not including cells. When such an array is incubated in a culture medium, cells disposed inside the two adjacent tubes each multiply, grow as a cell mass protruding from the tube, and come into contact with each other to form a contact surface. As long as a set of two adjacent tubes in which cells are disposed inside are sufficiently separated from other sets, there may be a plurality of sets in the array. That is, the array of this embodiment may be an array of pairs of adjacent tubes in which cells are disposed inside.

It is known that in the generation of living organisms, different types of stem cells form a contact surface with each other and form an organ with an interaction. Although it is difficult to reproduce such generation in vitro, it is possible to form and analyze a contact surface between different cells, using such an array.

Accordingly, in an embodiment, the present invention provides a method for culturing cells which includes a process of disposing cells inside two adjacent tubes of a plurality of tubes, and incubating an array in which the tubes around the two tubes do not contain cells in a culture medium, and as a result, the cells disposed inside the two adjacent tubes grow and come into contact with each other to form a contact surface.

### (Cells)

When the array of this embodiment includes cells, the cells are not particularly limited and include, for example, cell lines, primary cells, genetically modified cells, induced pluripotent stem cells (iPS cells), embryonic stem cells (ES cells), tissues stem cells, cells differentiated from stem cells, cell aggregates (spheroids) formed from these cells, and tissue pieces separated from a living body.

### (Adhesive Including CNP)

An adhesive including nanoparticles (hereinafter, sometimes referred to as “CNP”) in which surfaces are coated with a cationic water-soluble polymer will be described. Nanoparticles refer to particles having an average particle size of less than 1 μm. The average particle diameter of CNP is preferably 1 to 100 nm, more preferably 5 to 70 nm, and further preferably 20 to 50 nm. When the average particle size is within the aforementioned range, the anionic hydrogel tends to be more firmly bonded. Further, visible light can pass through the adhesive, and a part in which the adhesive is present can be made transparent.

Also, the charge on the surface of CNP is preferably, for example, about 10 to 50 mV.

The CNP is made of a cationic water-soluble polymer that coats the surface, and a core. The cationic water-soluble polymer may be a polymer having a cationic functional group. Examples of the cationic functional group include, but are not limited to, primary to quaternary amino groups and guanidine groups.

The cationic water-soluble polymer is a polymer obtained by polymerizing the aforementioned monomer having a cationic functional group (cationic monomer). Examples of the cationic monomer include vinylamine, allylamine, ethyleneimine, 3-(N,N-dimethylaminopropyl)-(meth)acrylamide, 3-(N,N-dimethylaminopropyl)-(meth)acrylate, aminostyrene, 2-(N,N-dimethylaminoethyl)-(meth)acrylamide, 2-(N,N-dimethylaminoethyl)-(meth)acrylate and salts thereof, halogenated diallyldialkylammonium and the like. These cationic monomers may be used alone or in combination of two or more.

The cationic water-soluble polymer may be a polymer obtained by copolymerizing the aforementioned cationic monomer with another monomer. The other monomer may be a hydrophilic monomer, or may be a hydrophobic monomer depending on a blending ratio.

The hydrophilic monomer may be one that is neutral in an aqueous solvent, and examples thereof include dimethylacrylamide, acrylic acid or methacrylic acid having a polyethylene glycol side chain, and the like. These may be used alone or in combination of two or more.

Examples of the hydrophobic monomer include those shown in (i) to (v) below. These may be used alone or in combination of two or more.

(i) Acrylic acid esters such as methyl acrylate, ethyl acrylate, isopropyl acrylate, n-butyl acrylate, and 2-ethylhexyl acrylate;

(ii) Methacrylic acid esters such as methyl methacrylate, ethyl methacrylate, isopropyl methacrylate, n-butyl methacrylate, isobutyl methacrylate, n-hexyl methacrylate, cyclohexyl methacrylate, lauryl methacrylate, and glycidyl methacrylate;

(iii) Aromatic olefins such as styrene and α-methylstyrene;

(iv) Vinyl esters such as vinyl acetate; and

(v) Vinyl nitriles such as acrylonitrile and methacrylonitrile.

Among these, the cationic water-soluble polymer is preferably polyethyleneimine obtained by polymerizing ethyleneimine or salts thereof. Polyethyleneimine is a polymer obtained by ring-opening polymerization of ethyleneimine by a known method. Further, a salt of polyethyleneimine is obtained by neutralizing some or all of the amino groups in polyethyleneimine with an acid. The acid used for neutralization may be an inorganic acid or an organic acid. Examples of the inorganic acid include hydrochloric acid, sulfuric acid, phosphoric acid, nitric acid and the like. Examples of the organic acid include acetic acid, formic acid, propionic acid and the like.

In CNP, a material forming the core is preferably a hydrophobic polymer. By the use of the hydrophobic polymer, a spherical CNP-including emulsion can be easily produced by a manufacturing method described below. The hydrophobic polymer is a polymer obtained by polymerizing a hydrophobic monomer. The hydrophobic monomer may be one in which a solubility in water at 25° C. is 10 g/dL or less, and specifically, the above-mentioned hydrophobic monomers that can be contained in the cationic water-soluble polymer constituent material are adopted.

Also, the hydrophobic polymer may be a polymer obtained by copolymerizing the aforementioned hydrophobic monomer and a crosslinkable monomer. Examples of the crosslinkable monomer include ethylene glycol di(meth)acrylate, hexanediol di(meth)acrylate, divinylbenzene, methylene bisacrylamide, trimethylolpropane tri(meth)acrylate, tetraallylethane, and the like. These may be used alone or in combination of two or more.

Among them, the hydrophobic polymer is preferably polystyrene obtained by polymerizing styrene.

The CNP can be obtained by emulsion polymerization of the aforementioned cationic monomer and hydrophobic monomer in an aqueous solvent in the presence of a radical polymerization initiator. In the emulsion polymerization, a formulation quantity of the cationic monomer with respect to the mass of the hydrophobic monomer is preferably 0.5 to 30% by mass or less, more preferably 0.5 to 15% by mass or less, and more preferably 0.5 to 5% by mass or less. When the blending quantity of the cationic monomer is equal to or less than the aforementioned lower limit value, and it is possible to obtain CNP more stably dispersed in the aqueous solvent. On the other hand, when the blending quantity of the cationic monomer is equal to or less than the aforementioned lower limit value, the CNP with a moderately positive charge tends to be obtained easily.

Examples of radical polymerization initiators include the following (i) to (v). These may be used alone or in combination of two or more.

(i) Oil-soluble azo compounds such as 2,2′-azobisisobutyronitrile, and 2,2′-azobis(2,4-dimethylvaleronitrile);

(ii) Water-soluble azo compounds such as 2,2′-azobis(2-amidinopropane) or its hydrochloride, 4,4′-azobis(4-cyanovaleric acid) or its alkali metal salts, 2,2′-azobis[2-(2-imidazolin-2-yl)propane] or its hydrochloride, and 2,2′-azobis[2-methyl-N-(2-hydroxyetol)propionamide];

(iii) Organic peroxides such as benzoyloxyperoxide and ditertiary butyl peroxide;

(iv) Inorganic peroxides such as potassium persulfate, sodium persulfate, and ammonium persulfate; and

(v): Redox initiators obtained by combining the aformentioned (iv) with a reducing substance (sodium sulfite, dimethylaminoethanol, and dimethylaminobenzoic acid).

Among them, the radical polymerization initiator is preferably the ones shown in the aforementioned (ii), which is 2,2′-azobis(2-amidinopropane) hydrochloride, 2,2′-azobis[2-(2-imidazoline-2-yl)propane] or a hydrochloride thereof, or 2,2′-azobis[2-methyl-N-(2-hydroxyetol)propionamide].

In the emulsion polymerization, the blending quantity of the radical polymerization initiator to the mass of the hydrophobic monomer can be, for example, 0.001 to 2% by mass or less. Further, the aqueous solvent used in the emulsion polymerization may be any solvent including water as a main component, and examples thereof include distilled water, deionized water, tap water, industrial water and the like.

Also, the emulsion polymerization is preferably a method called a soap-free emulsion polymerization that does not use a low-molecular-weight emulsifier. In this method, since the polymer forms fine particles in an aqueous solvent, by balancing the hydrophilicity and hydrophobicity between the cationic water-soluble polymer and the hydrophobic polymer, the CNP can be easily obtained.

In the emulsion polymerization, the total formulation quantity of the cationic water-soluble polymer and the hydrophobic polymer to the mass of the entire polymerization system is usually 1 to 70% by mass or less, preferably 10 to 60% by mass or less, and more preferably 20 to 60% by mass or less.

Further, as a system for producing CNP, for example, a method such as a batch type polymerization system, a continuous tube type polymerization system, and a semi-continuous polymerization system can be adopted. In the case of a batch type polymerization system, the procedures for adding the raw materials include, but are not limited to, those shown in (i) to (iii) below.

(i) A method in which a cationic monomer, a hydrophobic monomer, and a radical polymerization initiator are put together in a reaction tank to perform polymerization;

(ii) A method for polymerizing a cationic monomer, while individually adding a hydrophobic monomer and a radical polymerization initiator dropwise; and

(iii) A method for polymerizing a mixture of a hydrophobic monomer and a radical polymerization initiator, while dropping them into water including (cationic monomer).

The polymerization temperature and time are selected by the polymerizability of the monomer, the decomposition temperature and half-life of the initiator, etc. The polymerization temperature is usually 30 to 130° C., and preferably 50 to 100° C. The polymerization time is usually 1 to 10 hours.

The adhesive including CNP may be in the form of powder or liquid. Further, the adhesive including CNP may contain other components in addition to CNP to the extent that the cationic property of CNP is not impaired. Examples of other components include stabilizers, thickeners, preservatives and the like.

When the adhesive including CNP is in the form of liquid, it may contain, for example, an aqueous solvent. The aqueous solvent is not particularly limited, and examples thereof include water, physiological saline, physiological saline having a buffering effect and the like. Examples of the physiological saline having a buffering effect include phosphate buffered saline (PBS), Tris buffered saline (TBS), HEPES buffered saline and the like.

The adhesive including CNP may contain a water-soluble organic solvent, in addition to the water-based solvent. Examples of the water-soluble organic solvent include lower alcohols, acetone, dioxane, ethylene glycol and the like. The lower alcohol may be a monovalent alcohol having 1 to 3 carbon atoms, and specifically, for example, methanol, ethanol, propanol and the like are adopted.

### [Method for Transporting Non-Frozen Cells Alive]

In an embodiment, the present invention provides a method for transporting non-frozen cells alive, the method including a process of transporting a container accommodating the above-mentioned cell array and culture medium. As will be described later in examples, the method for this embodiment can transport non-frozen cells alive.

The cell array is preferably fixed on the substrate during the transportation period. The substrate may be glass, resin, metal or the like, but transparent glass or resin is easy to handle from the viewpoint of easy observation with a microscope. The above-mentioned CNP can be used for fixing the cell array onto the substrate.

During the transportation period, it is preferable to wrap the container including the cell array and the culture medium with a heat insulating material or the like to keep the temperature of the cell array and the culture medium at 33 to 36° C. In addition, it is preferable to enclose a CO₂ gas generating agent together with the container including the cell array and a culture medium to maintain a CO₂ concentration in a cell culture environment. It is confirmed that cells can be transported alive for at least 2 days, while keeping the functions of the cells alive.

### [Method for Manufacturing Array]

In an embodiment, the present invention provides a method for manufacturing an array, the method including a process of disposing a plurality of fibers including a target substance inside a tube made of anionic hydrogel to be in contact with each other so that axial directions are parallel to each other, and bonding the fibers with an adhesive including nanoparticles having a surface coated with a cationic water-soluble polymer to obtain a bundle of the fibers; a process of embedding the bundle in a support material; and a process of cutting the bundle together with the support material to obtain a section, in which the section is an array. The aforementioned array can be manufactured by the manufacturing method of this embodiment.

### (Fiber)

First, a method for manufacturing a fiber including the target substance will be described. A fiber is a fibrous structure including a target substance inside a tube made of anionic hydrogel. The anionic hydrogel is the same as that described above. Further, the target substance is also not particularly limited and is the same as that described above.

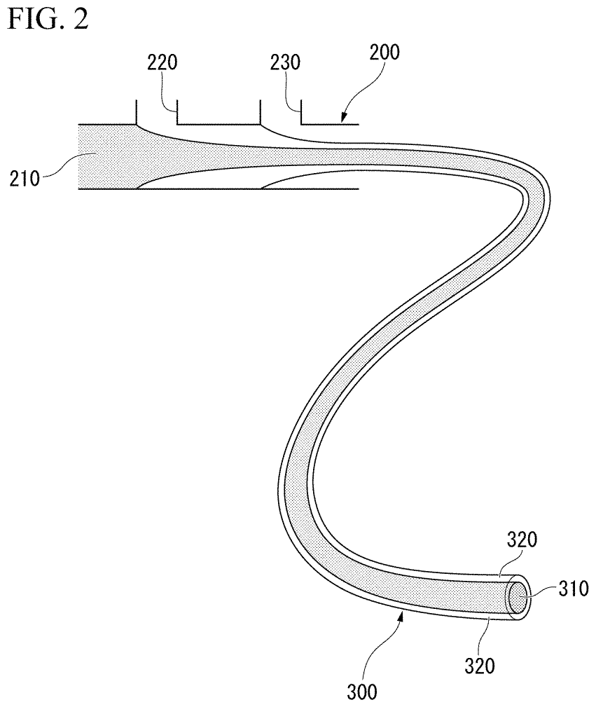

Although the method for manufacturing the fiber is not particularly limited, the fiber can be easily manufactured, for example, using a dual coaxial microfluidic device as shown in FIG. 2. A microfluidic device capable of injecting two fluids by dividing them into a core part and a shell part to be coaxial is also described in detail, for example, in Wonje Jeong, et al., Hydrodynamic microfabrication via “on the fly” photopolymerization of microscale fibers and tubes., Lab Chip, 2004, 4, 576-580 in FIG. 1.

FIG. 2 is a schematic view showing an example of a method for manufacturing the fiber 300. Here, the description will be provided for a case in which the target substance is cell, and a mixed solution of a culture medium, cells and an extracellular matrix (hereinafter referred to as “cell mixed solution”) is used as the material of the core part, and a sodium alginic acid solution before crosslinking is used as the material of the shell part. As the extracellular matrix, matrigel, collagen gel and the like can be used.

First, the cell mixture is introduced and injected from an inlet 210 of the microfluidic device 200. Further, the sodium alginic acid solution before crosslinking is introduced and injected from an inlet 220 of the microfluidic device 200. Further, a calcium chloride solution is introduced and injected from an inlet 230 of the microfluidic device 200. Then, the sodium alginic acid solution in the shell part gels, and it is possible to manufacture a fiber in which the core part 310 is a cell-including hydrogel and the shell part 320 is an alginic acid hydrogel.

An injection speed of the solution at the inlets 210 and 220 is not particularly limited, but may be about 10 to 500 μL/min when the diameter of the microfluidic device 200 is about 50 μm to 2 mm. By adjusting the injection speed of the solution at the inlets 210 and 220, the diameter of the core part and the coating thickness of the shell part can be adjusted appropriately. The injection speed of the solution at the inlet 230 is not particularly limited, but may be, for example, about 1 to 10 mL/min.

When manufacturing a hydrogel fiber including no target substance, a hydrogel including no target substance may be used instead of the aforementioned cell mixture. Further, when the target substance is a substance other than cells, a mixture of the target substance and a hydrogel material suitable for the target substance may be used as the material of the core part.

The method for manufacturing the fiber is not limited to that described above. For example, there is a case in which the target substance to be contained in the fiber may be rare and only a small amount can be produced. In such a case, it is effective to manufacture the fiber by the process of introducing the target substance into the tube made of anionic hydrogel through a funnel-type device. As a result, the amount of the target substance required for producing the fiber can be reduced.

### (Bundle of Fibers)

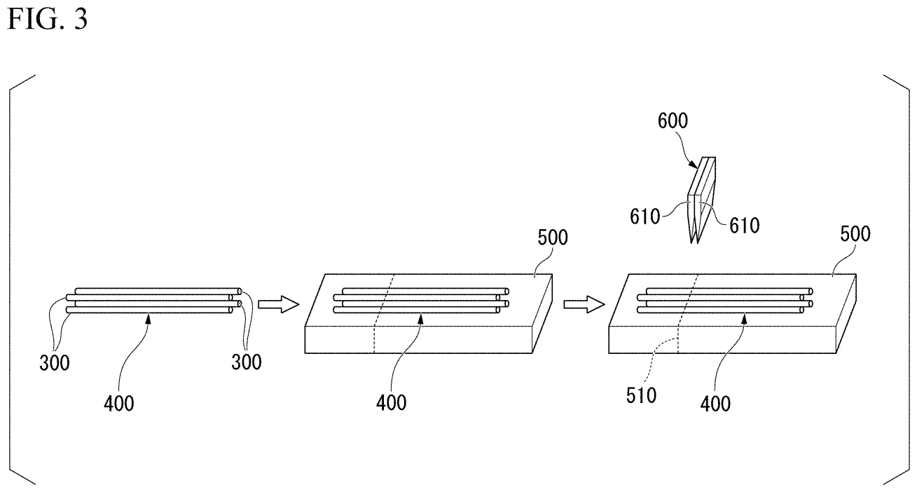

FIG. 3 is a schematic view showing the manufacturing method of this embodiment. First, a bundle of the fibers is manufactured. As shown in FIG. 3, first, a plurality of fibers 300 described above are disposed in contact with each other such that axial directions are parallel to each other. Subsequently, a bundle 400 of the fibers 300 is obtained by bonding with the above-mentioned adhesive including CNP.

Here, when manufacturing an array in which some tubes include the target substance inside and the remaining tubes do not contain the target substance, the bundle 400 may be manufactured, using the hydrogel fibers including no target substance instead of some of the fibers 300.

### (Support Material)

Subsequently, the bundle 400 of the fibers is embedded in the support material 500. Even if the bundle 400 is cut directly, the array cannot be cut out well. By embedding the bundle 400 in the support material 500 and cutting it together with the support material, the fibers forming the bundle 400 can be cut perpendicularly to the axial direction. Further, the layer including the target substance can be reliably cut to obtain an array.

A quotient (storage elastic modulus/loss elastic modulus) obtained by dividing a storage elastic modulus of the support material 500 by a loss elastic modulus is preferably larger than 10 and the storage elastic modulus is preferably 100 kPa or less.

As will be described later in the example, when the quotient (storage elastic modulus/loss elastic modulus) obtained by dividing the storage elastic modulus of the support material 500 by the loss elastic modulus is larger than 10 and the storage elastic modulus is 100 kPa or less, the array tends to be cut out accurately.

As the storage elastic modulus and loss elastic modulus of the support material 500, by performing a dynamic viscoelasticity test using a commercially available rheometer, values measured at 1 Hz may be used.

### (Cutting of Array)

Next, the bundle 400 is cut together with the support material 500 to obtain a section. For example, a cutter 600 in which two blades 610 for a microtome are stacked is pressed against a cutting line 510 of the support material 500, and the bundle 400 is cut together with the support material 500 to obtain a section. This section is the array. In the cutter 600, when the distance between the two blades 610 is 200 μm, the thickness of the array is 200 μm.

### EXAMPLES

The present invention will be described below by experimental examples, but the present invention is not limited to the following experimental examples.

### Experimental Example 1

### (Study of Support Material)

As mentioned above, when cutting a bundle of the fibers, it is necessary to embed the bundle in the support material. In this experimental example, the support material was examined. Alginic acid hydrogel was used as the support material.

By changing a ratio of gluconic acid, a ratio of mannuronic acid, a concentration of sodium alginic acid, and a concentration of calcium ions, which are the materials of the alginic acid hydrogel, five kinds of alginic acid hydrogels of GEL1 to 5 were produced and used as the support material, and the bundle of the fibers was embedded.

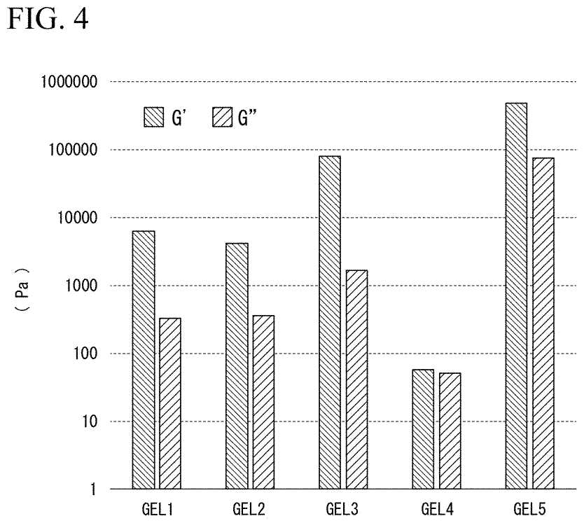

Subsequently, a dynamic viscoelasticity test of each support material was performed, using a rheometer (model “MCR302”, Anton Paar Co., Ltd.), and the storage elastic modulus (G′) and loss elastic modulus (G″) were measured at 1 Hz. FIG. 4 is a graph showing the measurement results of the storage elastic modulus (G′) and loss elastic modulus (G″) of each support material.

Also, the bundle of the fibers embedded in each support material was cut to cut the array. As a result, it was revealed that the array can be cut when GEL1 to 3 were used as the support material. On the other hand, when GEL4 and 5 were used as the support material, the array could not be cut.

From the above results, it was revealed that the array can be accurately cut, when the quotient obtained by dividing the storage elastic modulus (G′) of the support material by the loss elastic modulus (G″) was larger than 10 (G′/G″>10), and the storage elastic modulus (G′) was 100 kPa or less (G′≤100 kPa).

### Experimental Example 2

### (Production of Array)

### <Fluorescent Bead Array>

First, production of a fluorescent bead array was tried, using fluorescent beads as the target substance. As fluorescent beads, FluoSpheres Carboxylate-modified microspheres red, yellow-green, and blue (all are Thermo Fisher Scientific Inc.) were used.

Subsequently, a fiber including each fluorescent bead was produced. Next, the produced fiber was bonded with the adhesive agent including CNP mentioned above to produce the bundle. Subsequently, the bundle was embedded in the support material and cut with a blade for two microtomes stacked to obtain a fluorescent bead array.

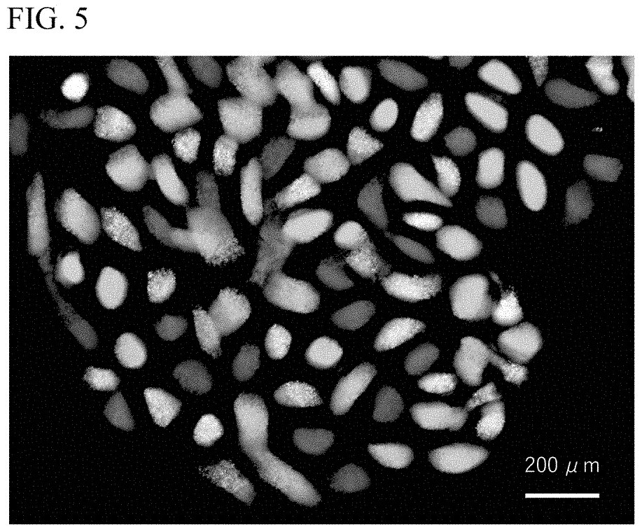

FIG. 5 is a photograph of the produced fluorescent bead array observed with a fluorescence microscope. As a result, it was confirmed that a fluorescent bead array observable with a microscope could be produced.

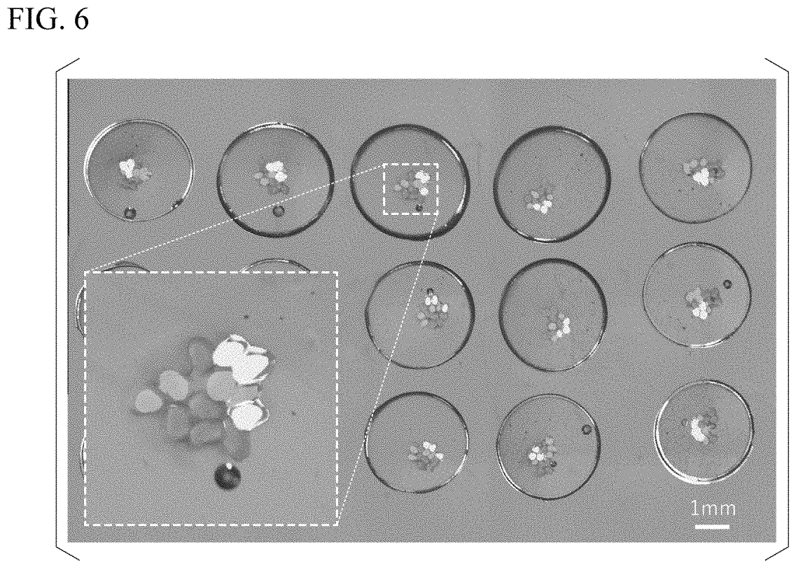

Also, FIG. 6 is a photograph in which a plurality of fluorescent bead arrays produced by the same method is observed with a fluorescent microscope. In a lower left of FIG. 6, an enlarged photograph of one of the fluorescent bead arrays is shown. As a result, it was confirmed that a large number of uniform fluorescent bead arrays could be produced by the method for this experimental example.

### <Cell Array>

Next, production of a cell array was tried, using cells as the target substance. A cell array was produced in the same manner as above except that HEK293T cells, which is a cell line derived from human embryonic kidney, were used instead of the fluorescent beads. Subsequently, it was examined whether the cells in the produced cell array were alive. Specifically, the cell array was dyed with a calcein AM (Takara Bio Inc.) which is a live cell dyeing reagent, an ethidium bromide (Takara Bio Inc.) which is a dead cell dyeing reagent, and a Hoechst 33342 (Thermo Fisher Scientific Inc.) which is a reagent for dyeing the nucleus, and observed with a fluorescence microscope.

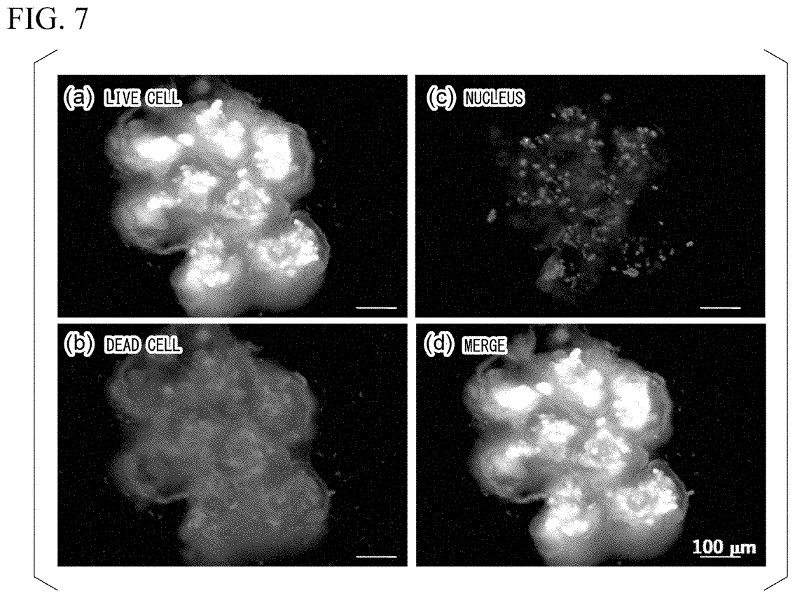

FIGS. 7(a) to 7(d) are photographs of the cell array observed with a fluorescence microscope. FIG. 7(a) is a photograph showing the result of detecting live cells. FIG. 7(b) is a photograph showing the results of detecting dead cells. FIG. 7(c) is a photograph showing the result of detecting nuclei. FIG. 7(d) is a photograph obtained by merging FIGS. 7(a) to 7(c). As a result, it was confirmed that the cells in the cell array were alive.

### Experimental Example 3

### (Chemical Substance Sensor Using Cells)

A muscarinic receptor was expressed in cells in the cell array, and the responsiveness to muscarine was examined.

Specifically, first, two days before the experiment, an expression vector of a human muscarinic acetylcholine receptor (a muscarinic receptor) was introduced into HEK293T cells, which is a cell line derived from human embryonic kidney, and was transiently expressed. Subsequently, a cell array was produced using these cells.

Subsequently, 100 mM muscarine was added to the culture medium of this cell array, and the inflow of calcium ions into the cells was measured. The inflow of calcium ions into cells was detected, by adding Fluo-8 and AM (AAT Bioquest Inc.), which is a calcium probe, to the culture medium and observing the fluorescence with a fluorescence microscope.

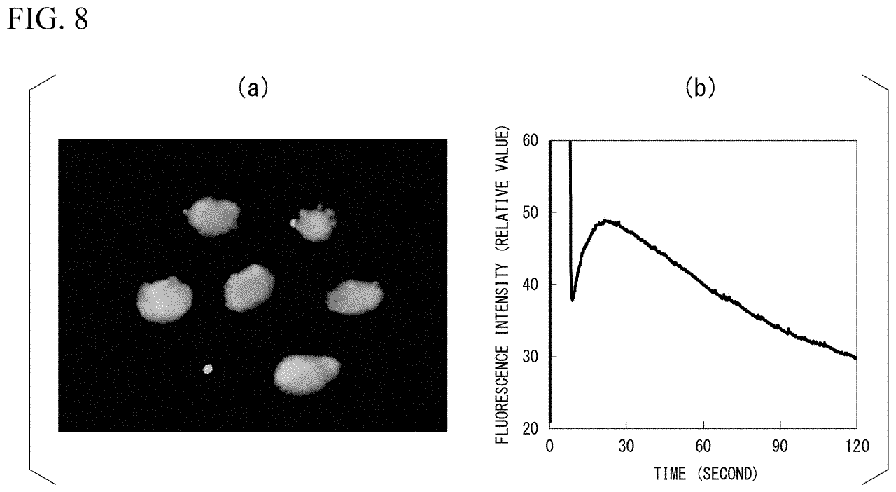

FIG. 8(a) is a fluorescence microphotograph showing an aspect in which the calcium probe emits fluorescence in response to muscarine added to the culture medium. FIG. 8(b) is a graph showing the results of measuring the fluorescence intensity of the calcium probe after the addition of muscarine in a time-dependent manner.

As a result, it was confirmed that an aspect in which cells expressing the muscarinic receptor respond to muscarine could be observed. This result shows that the cell array can be used as a chemical sensor.

### Experimental Example 4

### (Transportation of Cells)

Whether cells could be transported alive in the form of cell arrays was examined. Specifically, first, the cell array was fixed to a glass substrate with the above-mentioned adhesive including CNP and was accommodated in a 6-well plate together with a culture medium. Subsequently, the 6-well plate was put together with a CO₂ gas generating agent (trade name “Culture Pal”, Cosmo Bio Co., Ltd.) in a commercially available thermal insulation transporting box (Sanplatec Co., Ltd.) and packaged. In this thermal insulation transporting box, the inside temperature can be maintained at 33 to 36° C. for 150 hours or more, under the condition that the outside air temperature is 25° C.

Next, the thermal insulation transportation box was transported by courier, and made a round trip between Kanagawa prefecture and Kyoto prefecture. After two days of transportation, the state of the cell array was estimated.

Specifically, the cell array after transportation is dyed with a calcein AM (Takara Bio Inc.) which is a live cell dyeing reagent, and an ethidium bromide (Takara Bio Inc.) which is dead cell dyeing reagent, and observed with a fluorescence microscope.

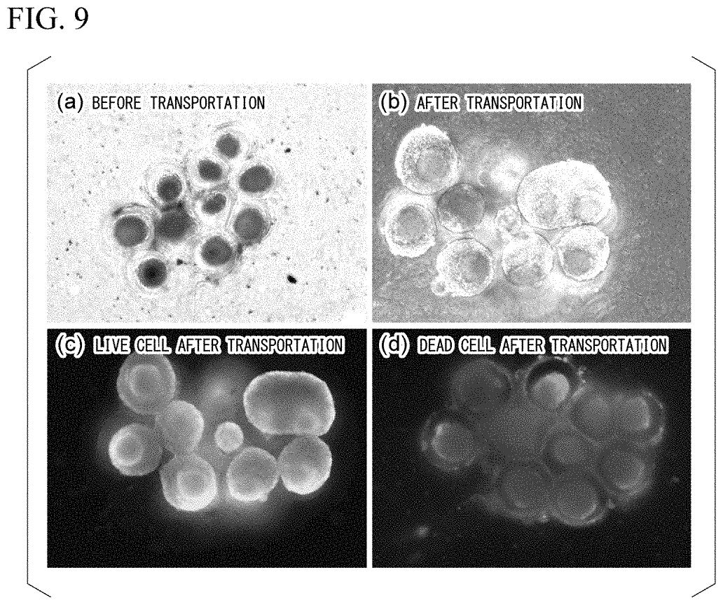

FIGS. 9(a) to 9(d) are photographs of cell arrays observed with a microscope. FIG. 9(a) is a microphotograph of the cell array observed before transportation. FIG. 9(b) is a microphotograph of the cell array observed after transportation. FIG. 9(c) is a photograph showing the results of detecting live cells after transportation. FIG. 9(d) is a photograph showing the result of detecting dead cells after transportation.

As a result, it was confirmed that a positional relationship of the cell array was maintained even after transportation. Further, it was confirmed that most of the cells survived after the transportation.

Also, as a result of performing the same experiment with a cell array produced with cells expressing muscarinic receptor stably, it was confirmed that the responsiveness to muscarine was maintained after transportation.

The aforementioned results show that by transporting in the form of a cell array, it is possible to transport cells of a non-frozen state, while keeping them alive and the function of the cells.

### INDUSTRIAL APPLICABILITY

According to the present invention, a new array can be provided.

### REFERENCE SIGNS LIST

100: Array, 120: Tube, 110: Target substance, 130: Surface, 200: Microfluidic device, 210, 220, 230: Inlet, 310: Core part, 320: Shell part, 300: Fiber, 400: Bundle, 500: Support material, 510: Cutting line, 610: Blade, 600: Cutter.

1. An array comprising:
a plurality of tubes disposed in contact with each other such that axial directions are parallel to each other; and
a target substance disposed inside at least one of the plurality of tubes,
wherein the plurality of tubes are made of anionic hydrogel, and surfaces on which the plurality of tubes are in contact with each other are bonded with an adhesive including nanoparticles having surfaces coated with a cationic water-soluble polymer.

2. The array according to claim 1, wherein some of the plurality of tubes include the target substance inside, and the remaining tubes include no target substance.

3. The array according to claim 1, wherein the target substance is a cell.

4. The array according to claim 3, wherein the cells include a cell which respond to a chemical substance.

5. The array according to claim 3, wherein cells are disposed inside two adjacent tubes of the plurality of tubes, and tubes around the two tubes include no cells.

6. A cell culture method comprising:
incubating the array according to claim 5 in a culture medium,
wherein, as a result, the cells disposed inside two adjacent tubes each grow and come into contact each other to form a contact surface.

7. A method for transporting non-frozen cells alive, the method comprising:
transporting a container which accommodates the array according to claim 3 and a culture medium.

8. A method for manufacturing an array, the method comprising:
disposing a plurality of fibers including a target substance inside a tube made of anionic hydrogel to be in contact with each other so that axial directions are parallel to each other, and bonding the fibers with an adhesive including nanoparticles having a surface coated with cationic water-soluble polymer to obtain a bundle of the fibers;
embedding the bundle in a support material; and
cutting the bundle together with the support material to obtain a section,
wherein the section is an array.

9. The method for manufacturing an array according to claim 8, wherein a quotient (a storage elastic modulus/a loss elastic modulus) obtained by dividing a storage elastic modulus of the support material by a loss elastic modulus is larger than 10, and the storage elastic modulus is 100 kPa or less.

10. The method for manufacturing an array according to claim 8, wherein the fiber is manufactured by a process of introducing the target substance through a funnel-type device into a tube made of anionic hydrogel.

11. The method for manufacturing an array according to claim 8, wherein the target substance is a cell.
